(19) United States
(12) Patent Application Publication
(10) Pub. No.: US 2026/0335292 A1
(43) Pub. Date: Oct. 29, 2026

(54) ANTI-AGING COMPOSITION COMPRISING OXAZOLE DERIVATIVE

(71) Applicants: INCHEON NATIONAL UNIVERSITY RESEARCH & BUSINESS FOUNDATION, Incheon (KR); KOREA UNIVERSITY RESEARCH AND BUSINESS FOUNDATION, SEJONG CAMPUS, Sejong (KR)

(72) Inventors: Joon Tae PARK, Incheon (KR); Yun Haeng LEE, Seoul (KR); Hyung Wook KWON, Seoul (KR); Young Joo BYUN, Daejeon (KR); Hyun Woong LIM, Seoul (KR)

(73) Assignees: INCHEON NATIONAL UNIVERSITY RESEARCH & BUSINESS FOUNDATION, Incheon (KR); KOREA UNIVERSITY RESEARCH AND BUSINESS FOUNDATION, SEJONG CAMPUS, Sejong (KR)

(21) Appl. No.: 19/554,599

(22) Filed: Mar. 2, 2026

Related U.S. Application Data
(63) Continuation of application No. PCT/KR2025/000594, filed on Jan. 10, 2025.

(30) Foreign Application Priority Data
Aug. 16, 2024 (KR) 10-2024-0110058
Dec. 18, 2024 (KR) 10-2024-0190507

(51) Int. Cl. A61K 31/421; A61K 8/49; A61P 39/06; A61P 43/00; A61Q 19/08

(52) U.S. Cl. CPC A61K 31/421; A61K 8/49; A61P 39/06; A61P 43/00; A61Q 19/08; A61K 2800/522

(57) ABSTRACT

The present invention relates to an anti-aging composition, and more particularly, to an anti-aging composition comprising an oxazole derivative represented by [Chemical Formula 1] as an active ingredient, wherein the aging is induced by mitochondrial dysfunction, and the composition reduces reactive oxygen species (ROS) in senescent fibroblasts,

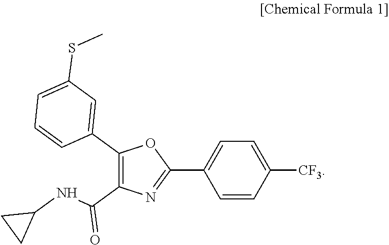 [SMILES: CSc1cccc(-c2oc(-c3ccc(C(F)(F)F)cc3)nc2C(=O)NC2CC2)c1]

### CROSS-REFERENCE TO RELATED APPLICATION

This application is a continuation application of PCT International Patent Application No. PCT/KR2025/000594 filed on Jan. 10, 2025, which claims priority to Korean Patent Application Nos. 10-2024-0110058 filed on Aug. 16, 2024 and 10-2024-0190507 filed on Dec. 18, 2024, which are all hereby incorporated by reference in their entirety.

### BACKGROUND

The present invention relates to an anti-aging composition, and more particularly, to an anti-aging composition comprising an oxazole derivative represented by [Chemical Formula 1] as an active ingredient:

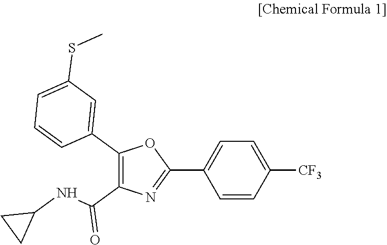 [SMILES: CSc1cccc(-c2oc(-c3ccc(C(F)(F)F)cc3)nc2C(=O)NC2CC2)c1]

Research on aging has been continuously conducted from the past to the present. This is because aging is one of the disease-causing factors that can induce various diseases. A stage in which the cessation of cell division persists permanently is defined as cellular senescence, which is regarded as a fundamental mechanism of aging and has been highlighted as a cause of aging-related diseases such as cancer, atherosclerosis, and degenerative osteoarthritis. In the early stages of research on aging, biologists speculated that the aging of living tissues results from the suppression of repair mechanisms of surrounding cells by cells that no longer undergo differentiation. Such cells that no longer differentiate are now referred to as senescent cells (SNCs). Senescent cells are known not only to fail to participate in the repair of damaged cellular tissues, but also to induce migration of inflammatory cells, remodeling of the extracellular matrix, undesirable cell death, and fibrosis due to substances secreted from the senescent cells. These senescent cells naturally accumulate with aging, thereby leading to the accumulation of damage in cellular tissues.

Current technologies are capable of selectively eliminating senescent cells, thereby providing significant potential for the development of methods for preventing and treating numerous aging-related diseases. Broadly, therapeutic approaches include senolysis, immune-mediated elimination of senescent cells, and neutralization of the senescence-associated secretory phenotype (SASP). Among these, permanent removal of senescent cells not only induces a sustained reduction of SASP, but also reduces the risk of cancer development that may occur if senescent cells survive. However, there are considerations that must be addressed prior to applying such technologies. First, it is necessary to determine from which cells and by which methods senescent cells should be induced, and to which disease models they should be applied. In addition, in actual mouse experiments, senescent cells may be induced by applying stress, by surgical procedures, or by using aged mice, and it is also important to determine which of these methods should be employed. Nevertheless, research on aging has yet to achieve definitive outcomes, and accordingly, there remains a need for the development of novel materials or methods capable of inhibiting aging.

### SUMMARY

Compounds that improve aging may be applied across a wide range of fields. Aging is associated with various factors related to health problems. Anti-aging drugs may be used in the health and anti-aging fields and may help prevent diseases, enhance immunity, and improve metabolic functions through their pharmacological efficacy. In addition, anti-aging drugs may be used in the cosmetic and aging management industries, serving as solutions for cosmetic and aging-related issues such as improvement of skin health, reduction of wrinkles, restoration of elasticity, and hair regeneration. Anti-aging drugs also have significant potential for application in the medical field, where they may be utilized in research on longevity as well as in the prevention and treatment of aging-related diseases, and in studies investigating the association between aging and age-related disorders. Furthermore, anti-aging drugs may be used in the fields of sports and performance enhancement, where they may contribute to improved physical strength, increased muscle power, and enhanced recovery in athletes, and may also help prevent performance decline due to aging.

Aging is characterized by an increase in oxygen radicals, i.e., reactive oxygen species (ROS), generated in the mitochondria of cells. The same cause also applies to skin aging. In the present invention, it was confirmed that, in senescent fibroblasts, the oxazole derivative represented by [Chemical Formula 1] not only significantly reduces the ROS levels of senescent fibroblasts, but also improves cell viability and suppresses apoptosis. In addition, meaningful recovery of other aging markers by the oxazole derivative was confirmed.

In summary, the present invention demonstrates that a senomorphic mechanism of the oxazole derivative represented by [Chemical Formula 1] operates in senescent fibroblasts to reduce ROS and restore mitochondrial function.

According to one embodiment of the present invention, the present invention provides an anti-aging composition comprising an oxazole derivative represented by the following [Chemical Formula 1] as an active ingredient;

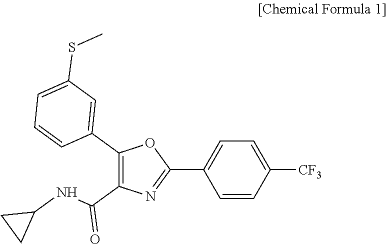 [SMILES: CSc1cccc(-c2oc(-c3ccc(C(F)(F)F)cc3)nc2C(=O)NC2CC2)c1]

The aging is induced by mitochondrial dysfunction.

The composition reduces reactive oxygen species (ROS) in senescent fibroblasts.

The composition enhances intracellular mitochondrial respiration.

The composition has a selective cytotoxic effect on senescent cells at a concentration of 4 μM or higher.

The composition restores autophagy system and increases glycolytic metabolic activity.

The composition reduces ROS in senescent cells.

The composition reduces lipofuscin in senescent cells.

The composition restores cellular telomeres.

The composition reduces β-galactosidase (SA-β-gal) accumulated within cells.

The composition reduces the proportion of G1/G0 phase and increases the proportions of S phase and G2/M phase in cell cycle, thereby activating the cell cycle.

According to another embodiment of the present invention, the present invention provides a pharmaceutical composition for preventing or treating cellular senescence-related diseases induced by mitochondrial dysfunction, the pharmaceutical composition comprising an oxazole derivative represented by the following [Chemical Formula 1] or a pharmaceutically acceptable salt thereof.

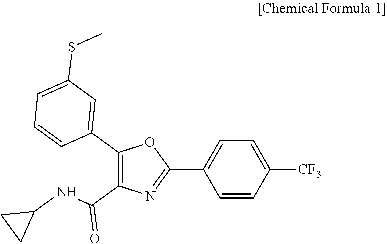 [SMILES: CSc1cccc(-c2oc(-c3ccc(C(F)(F)F)cc3)nc2C(=O)NC2CC2)c1]

The cellular senescence-related disease induced by mitochondrial dysfunction may be selected from the group consisting of Friedreich's ataxia (FRDA), renal tubular acidosis, Parkinson's disease, Alzheimer's disease, amyotrophic lateral sclerosis (ALS), Huntington's disease, pervasive developmental disorder, hearing loss, deafness, diabetes, obesity, diabetic ulcer, insulin resistance, and hepatic steatosis (steatohepatitis), but is not limited thereto.

The present invention provides an anti-aging cosmetic composition comprising, as an active ingredient, an oxazole derivative represented by the following [Chemical Formula 1];

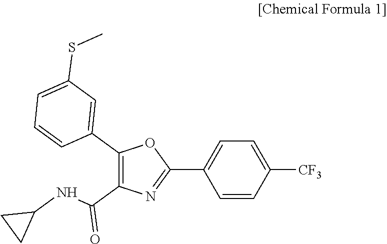 [SMILES: CSc1cccc(-c2oc(-c3ccc(C(F)(F)F)cc3)nc2C(=O)NC2CC2)c1]

The oxazole derivative represented by [Chemical Formula 1], which is the active ingredient of the present invention, reduces reactive oxygen species (ROS) in senescent fibroblasts, enhances intracellular mitochondrial respiration, and exhibits a selective cytotoxic effect on senescent cells at a concentration of 4 μM or higher. In addition, the oxazole derivative reduces intracellular ROS, lipofuscin, and β-galactosidase (SA-β-gal) in senescent cells, restores cellular telomeres, decreases the proportion of G1/G0 phase and increases the proportions of S phase and G2/M phase in the cell cycle, restores the autophagy system, and increases glycolytic metabolic activity, thereby inhibiting mitochondrial dysfunction-induced cellular senescence. Accordingly, the oxazole derivative represented by [Chemical Formula 1] may be used as a composition for preventing or treating cellular senescence-related diseases induced by mitochondrial dysfunction.

### BRIEF DESCRIPTION OF THE DRAWINGS

FIGS. 1 and 2 are images showing the results of ROS assessment of an oxazole library using high-throughput screening.

FIG. 3 depicts a graph showing the effect of KB2779 on the reduction of cell proliferation in senescent fibroblasts.

FIG. 4 depicts a graph comparing programmed cell death in young fibroblasts and senescent fibroblasts treated with KB2779.

FIG. 5 depicts a graph illustrating the effect of KB2779 on improving senescence-related phenotypes.

FIG. 6 depicts a graph showing the effect of KB2779 on the activation of the cell cycle in senescent fibroblasts.

FIG. 7 depicts a graph showing the recovery of autophagy function in senescent cells under the influence of KB2779.

FIG. 8 depicts a graph illustrating the metabolic regulation effect of KB2779 in senescent fibroblasts.

### DETAILED DESCRIPTION OF THE INVENTION

According to one embodiment of the present invention, the present invention provides an anti-aging composition comprising, as an active ingredient, an oxazole derivative represented by the following [Chemical Formula 1].

According to another embodiment of the present invention, the present invention provides a pharmaceutical composition for preventing or treating cellular senescence-related diseases induced by mitochondrial dysfunction, the pharmaceutical composition comprising an oxazole derivative represented by the following [Chemical Formula 1], or a pharmaceutically acceptable salt thereof.

According to another embodiment of the present invention, the present invention provides an anti-aging cosmetic composition comprising, as an active ingredient, an oxazole derivative represented by the following [Chemical Formula 1].

The inventors have confirmed that the oxazole derivative represented by [Chemical Formula 1], which is the active ingredient of the present invention, reduces reactive oxygen species (ROS) in senescent fibroblasts, enhances intracellular mitochondrial respiration, exhibits a selective cytotoxic effect on senescent cells at a concentration of 4 M or higher, reduces intracellular ROS, lipofuscin, and β-galactosidase (SA-β-gal) in senescent cells, restores cellular telomeres, decreases the proportion of G1/G0 phase and increases the proportions of S phase and G2/M phase in the cell cycle, restores the autophagy system, and increases glycolytic metabolic activity, thereby inhibiting mitochondrial dysfunction-induced cellular senescence. Accordingly, the oxazole derivative represented by [Chemical Formula 1] can be used as a composition for preventing or treating cellular senescence-related diseases or as a cosmetic composition for suppressing skin aging.

Accordingly, according to one embodiment of the present invention, the present invention provides an anti-aging composition comprising an oxazole derivative represented by the following [Chemical Formula 1] as an active ingredient.

According to another embodiment of the present invention, the present invention provides a pharmaceutical composition for preventing or treating cellular senescence-related diseases, comprising an oxazole derivative represented by the following [Chemical Formula 1], or a pharmaceutically acceptable salt thereof.

According to another embodiment of the present invention, the present invention provides a food composition for preventing or improving cellular senescence-related diseases, comprising an oxazole derivative represented by the following [Chemical Formula 1], or a pharmaceutically acceptable salt thereof.

According to yet another embodiment of the present invention, the present invention provides an anti-aging cosmetic composition, specifically a cosmetic composition for suppressing skin aging, comprising an oxazole derivative represented by the following [Chemical Formula 1] as an active ingredient.

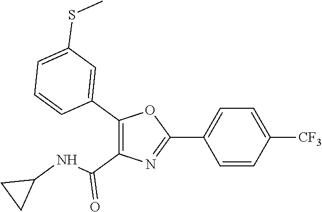 [SMILES: CSc1cccc(-c2oc(-c3ccc(C(F)(F)F)cc3)nc2C(=O)NC2CC2)c1]

The aging is induced by mitochondrial dysfunction.

The cellular senescence-related diseases induced by mitochondrial dysfunction may be selected from the group consisting of Friedreich's ataxia (FRDA), renal tubular acidosis, Parkinson's disease, Alzheimer's disease, amyotrophic lateral sclerosis (ALS), Huntington's disease, pervasive developmental disorder, hearing loss, deafness, diabetes, obesity, diabetic ulcer, insulin resistance, and hepatic steatosis (steatohepatitis), but are not limited thereto.

The pharmaceutical composition for preventing or treating the cellular senescence-related diseases may be prepared as a conventional formulation by selecting one or two pharmaceutically acceptable carriers and/or one or two additives in an effective amount of the active ingredient.

The carrier may be selected from one or more of diluents, lubricants, binders, disintegrants, sweeteners, stabilizers, and preservatives, and the additive may be selected from one or more of flavors, vitamins, and antioxidants.

In the present invention, any pharmaceutically acceptable carriers and additives may be used. Specifically, preferred diluents include lactose monohydrate, trehalose, cornstarch, soybean oil, microcrystalline cellulose, or D-mannitol; preferred lubricants include magnesium stearate or talc; preferred binders include polyvinylpyrrolidone (PVP) or hydroxypropylcellulose (HPC). Preferred disintegrants include carboxymethylcellulose calcium (Ca-CMC), sodium starch glycolate, polacrylin potassium, or cross-linked polyvinylpyrrolidone; preferred sweeteners include sucrose, fructose, sorbitol, or aspartame; preferred stabilizers include carboxymethylcellulose sodium (Na-CMC), O-cyclodextrin, white bee's wax, or xanthan gum; and preferred preservatives include methyl p-hydroxybenzoate (methylparaben), propyl p-hydroxybenzoate (propylparaben), or potassium sorbate. However, the present invention is not limited to these specific examples.

The pharmaceutical composition of the present invention may be administered to a patient as a single dose, or may be administered in a fractionated treatment protocol in which multiple doses are given over an extended period. As used herein, a “pharmaceutically effective amount” refers to an amount sufficient to elicit a response greater than that observed in a negative control, and preferably, an amount sufficient to prevent or treat an inflammatory disease. The pharmaceutically effective amount may vary depending on factors such as the type and severity of the disease, the age, weight, health condition, and sex of the patient, the route of administration, and the treatment duration.

The composition of the present invention may be formulated in various dosage forms according to the route of administration using pharmaceutically acceptable carriers by methods known in the art. As used herein, “pharmaceutically acceptable” refers to a composition that is physiologically acceptable, does not inhibit the activity of the active ingredient, and is non-toxic, generally avoiding allergic or similar reactions such as gastrointestinal disturbances or dizziness when administered to humans. The composition may be formulated according to the route of administration, which may be oral or non-oral, without limitation.

The food composition may include beverages (including tea or alcoholic beverages), fruits and processed fruit products (e.g., canned fruits, bottled fruits, jams, marmalades), fish, meat and processed meat products (e.g., ham, sausages, corned beef), breads and noodles (e.g., udon, buckwheat noodles, ramen, spaghetti, macaroni), fruit juices, various drinks, cookies, taffy, dairy products (e.g., butter, cheese), edible vegetable oils, margarine, plant proteins, retort foods, frozen foods, and various seasonings (e.g., soybean paste, soy sauce, sauces), among others.

In addition, the food composition may be a health functional food, which may be formulated as tablets, pills, powders, granules, capsules, or liquid forms. These formulations may include one or more carriers, diluents, excipients, and additives.

Skin aging can be classified into (i) intrinsic aging, which occurs naturally due to a decline in the structure and physiological functions of the skin with age, independent of environmental changes, and (ii) extrinsic aging, which is caused by external factors such as ultraviolet (UV) radiation, dry air, reactive oxygen species (ROS), and stress. Intrinsic aging manifests clinically as mild features such as fine wrinkles, dry skin, and decreased elasticity, whereas extrinsic aging exhibits clinical features including deep and coarse wrinkles along with fine wrinkles. Therefore, decreased skin elasticity or wrinkles are representative symptoms of skin aging. Accordingly, the skin aging described herein may be characterized by the occurrence of wrinkles or a reduction in skin elasticity.

In other words, the prevention or improvement of skin aging may involve wrinkle reduction, enhancement of skin elasticity, antioxidant activity, or anti-inflammatory effects, but is not limited thereto.

The cosmetic composition of the present invention can include ingredients commonly used in cosmetic compositions, such as fatty substances, organic solvents, solubilizers, thickeners and gelling agents, emollients, antioxidants, suspending agents, stabilizers, foaming agents, fragrances, surfactants, water, ionic or nonionic emulsifiers, fillers, metal ion sequestering agents and chelating agents, preservatives, vitamins, UV blockers, humectants, essential oils, dyes, pigments, hydrophilic or lipophilic actives, lipid vesicles, and other conventional excipients or carriers in amounts conventionally used.

The cosmetic composition of the present invention may be prepared in any formulation commonly manufactured in the industry, such as emulsions, creams, toners, masks, foundations, lotions, serums, or hair cosmetics. Specifically, the cosmetic composition may be formulated as skin lotion, skin softener, skin toner, astringent, lotion, milk lotion, moisture lotion, nourishing lotion, massage cream, nourishing cream, moisture cream, hand cream, foundation, essence, nourishing essence, mask, soap, cleansing foam, cleansing lotion, cleansing cream, body lotion, or body cleanser.

When the formulation is a paste, cream, or gel, carrier components may include animal or plant fibers, wax, paraffin, starch, tragacanth, cellulose derivatives, polyethylene glycol, silicone, bentonite, silica, talc, or zinc oxide. When the formulation is a powder or spray, carrier components may include lactose, talc, silica, aluminum hydroxide, calcium silicate, or polyamide powder. In particular, spray formulations may additionally include propellants such as chlorofluorohydrocarbon, propane/butane, or dimethyl ether.

When the formulation is a solution or emulsion, carrier components may include solvents, solubilizers, or emulsifiers, for example, water, ethanol, isopropanol, ethyl carbonate, ethyl acetate, benzyl alcohol, benzyl benzoate, propylene glycol, 1,3-butylene glycol, glycerol aliphatic esters, polyethylene glycol, or sorbitan fatty acid esters. When the formulation is a suspension, carrier components may include liquid diluents such as water, ethanol, or propylene glycol, suspending agents such as ethoxylated isostearyl alcohol, polyoxyethylene sorbitol esters, and polyoxyethylene sorbitan esters, and excipients such as microcrystalline cellulose, aluminum metahydroxide, bentonite, agar, or tragacanth.

When the formulation is a surfactant-containing cleansing composition, carrier components may include aliphatic alcohol sulfates, aliphatic alcohol ether sulfates, sulfosuccinate monoesters, isethionates, imidazolinium derivatives, methyltaurates, sarcosinates, fatty acid amide ether sulfates, alkylamidobetaines, aliphatic alcohols, fatty acid glycerides, fatty acid diethanolamides, vegetable oils, lanolin derivatives, or ethoxylated glycerol fatty acid esters.

The present invention is described in more detail through the following examples. However, the present invention is not limited to these examples and may be embodied in any form. The examples are provided solely to aid in understanding the present invention.

### EXPERIMENTS

### 1. Synthesis of an Oxazole Derivative

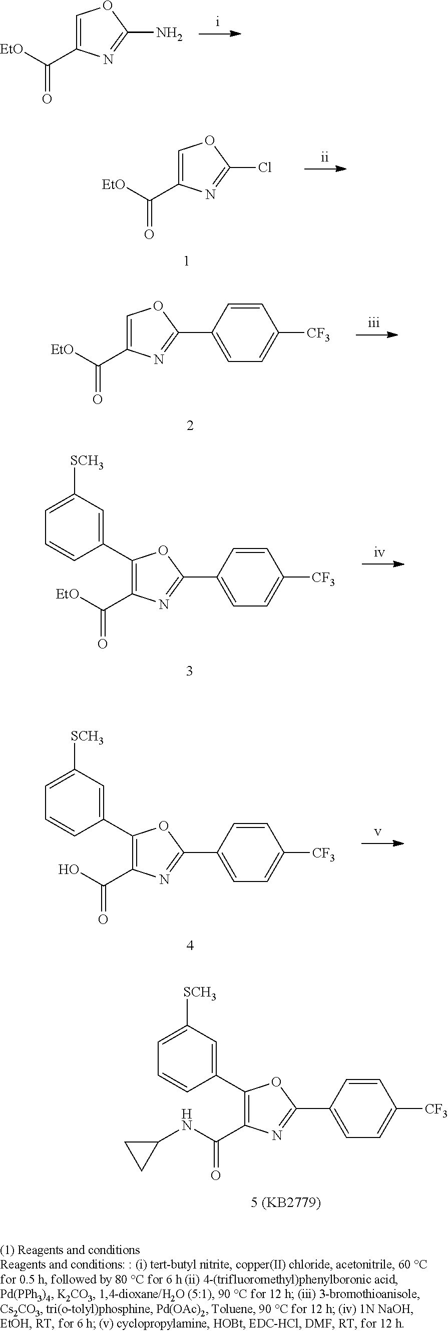 [SMILES: CCOC(=O)c1coc(N)n1 ; CCOC(=O)c1coc(Cl)n1 ; CCOC(=O)c1coc(-c2ccc(C(F)(F)F)cc2)n1 ; CCOC(=O)c1nc(-c2ccc(C(F)(F)F)cc2)oc1-c1cccc(SC)c1 ; CSc1cccc(-c2oc(-c3ccc(C(F)(F)F)cc3)nc2C(=O)O)c1 ; CSc1cccc(-c2oc(-c3ccc(C(F)(F)F)cc3)nc2C(=O)NC2CC2)c1]

### Ethyl 2-chlorooxazole-4-carboxylate) (1)

Ethyl 2-aminooxazole-4-carboxylate (2.0 g, 12.8 mmol) was added to a solution of tert-butyl nitrite (2.28 mL, 19.2 mmol, 1.5 eq) and copper(II) chloride (2.60 g, 19.2 mmol, 1.5 eq) in acetonitrile (60 mL) at 60 °C. The mixture was stirred under an argon atmosphere for 30 minutes, then stirred at 80 °C for 6 hours. The solution was cooled to room temperature and partitioned between dichloromethane, water, and concentrated hydrochloric acid. The aqueous layer was further extracted with dichloromethane, and the collected organic layers were washed with brine, dried over magnesium sulfate (MgSO₄), and concentrated under reduced pressure. The residue was purified by silica gel column chromatography (Hexane/Et₂O=11:1 to 9:1, v/v) to afford Compound 1 (702 mg, 62%) as a white solid.

R_{f} 0.41 (Hexane/Et₂O=3:1, v v). ¹H NMR (600 MHz, CDCl₃): δ 8.18 (s, 1H), 4.39 (q, J=7.2 Hz, 2H), 1.38 (t, J=7.2 Hz, 3H)

### Ethyl 2-(4-(trifluoromethyl)phenyl)oxazole-4-carboxylate (2)

Compound 1 (630 mg, 3.59 mmol), 4-(trifluoromethyl)phenylboronic acid (887 mg, 4.67 mmol, 1.3 eq), tetrakis(triphenylphosphine)palladium (0) (208 mg, 0.18 mmol, 0.05 eq), and potassium carbonate (1.49 g, 10.77 mmol, 3.0 eq) were dissolved in a degassed 1,4-dioxane/water mixture (5:1, 25 mL) under an argon atmosphere, and the reaction mixture was stirred at 90 °C for 12 hours. After cooling to room temperature, the reaction mixture was partitioned between ethyl acetate and water. The organic layer was washed with brine, dried over magnesium sulfate (MgSO₄), and concentrated under reduced pressure. The concentrate was purified by silica gel column chromatography (Hexane/Ethyl acetate=9:1 to 5:1, v/v) to afford Compound 2 (586 mg, 57%) as a white solid.

R_{f} 0.48 (Hexane/Ethyl acetate=4:1, v v). ¹H NMR (600 MHz, CDCl₃): δ 8.32 (s, 1H), 8.25 (d, J=8.3 Hz, 2H), 7.75 (d, J=8.3 Hz, 2H), 4.45 (q, J=7.1 Hz, 2H), 1.42 (t, J=7.1 Hz, 3H)

### Ethyl 5-(3-(methylthio)phenyl)-2-(4-(trifluoromethyl)phenyl)oxazole-4-carboxylate) (3)

Compound 2 (586 mg, 2.05 mmol), 3-bromothioanisole (330 μL, 2.46 mmol, 1.2 eq), palladium acetate (92 mg, 0.41 mmol, 0.2 eq), tri(o-tolyl)phosphine (125 mg, 0.41 mmol, 0.2 eq), cesium carbonate (1.0 g, 4.10 mmol, 1.5 eq), and toluene (20 mL) were stirred at 90 °C for 12 hours under an argon atmosphere. The cooled mixture was partitioned between ethyl acetate and water. The organic layer was washed with brine, dried over magnesium sulfate (MgSO₄), and concentrated under reduced pressure. The concentrate was purified by silica gel column chromatography (Hexane/Ethyl acetate=9:1 to 5:1, v/v) to afford Compound 3 (471 mg, 56%) as a yellow solid.

R_{f} 0.53 (Hexane/Ethyl acetate=4:1, v/v). ¹H NMR (600 MHz, CDCl₃): δ 8.28 (d, J=8.2 Hz, 2H), 8.04 (s, 1H), 7.86 (d, J=7.7 Hz, 1H), 7.76 (d, J=8.2 Hz, 2H), 7.43 (t, J=7.8 Hz, 1H), 7.38 (d, J=8.0 Hz, 1H), 4.47 (q, J=7.1 Hz, 2H), 2.57 (s, 3H), 1.43 (t, J=7.1 Hz, 3H)

### 5-(3-(methylthio)phenyl)-2-(4-(trifluoromethyl)phenyl)oxazole-4-carboxylic acid) (4)

1 N sodium hydroxide solution (1.47 mL, 1.47 mmol, 3.0 eq) was added to a solution of Compound 3 (200 mg, 0.49 mmol) in ethanol (30 mL). The reaction mixture was stirred at room temperature for 6 hours. Ethanol was evaporated, and 3 N hydrochloric acid was added until no further precipitation occurred. The precipitate was filtered and dried to afford Compound 4 (181 mg, 98%) as a white solid, which was used in the next step without further purification.

¹H NMR (600 MHz, DMSO-d₆): δ 8.32 (d, J=8.2 Hz, 2H), 8.09 (s, 1H), 7.96 (d, J=8.2 Hz, 2H), 7.89 (d, J=7.7 Hz, 1H), 7.50 (t, J=7.8 Hz, 1H), 7.43 (d, J=8.0 Hz, 1H), 2.55 (s, 3H)

### N-cyclopropyl-5-(3-(methylthio)phenyl)-2-(4-(trifluoromethyl)phenyl)oxazole-4-carboxamide) (5, KB2779)

Compound 4 (150 mg, 0.40 mmol), hydroxybenzotriazole hydrate (HOBt) (85 mg, 0.63 mmol, 1.6 eq), 1-ethyl-3-(3-dimethylaminopropyl)carbodiimide (EDC) (121 mg, 0.63 mmol, 1.6 eq), 1- and cyclopropylamine (33 μL, 0.47 mmol, 1.2 eq) in dimethylformamide (5 mL) were added. The solution was stirred at room temperature for 12 hours, and the solvent was removed. The residue was partitioned between ethyl acetate and water. The organic layer was washed with brine, dried over magnesium sulfate (MgSO₄), and concentrated under reduced pressure. The concentrate was purified by silica gel column chromatography (Hexane/Ethyl acetate=9:1 to 6:1, v/v) to afford Compound 5 (58 mg, 35%) as a white solid.

R_{f} 0.37 (Hexane/Ethyl acetate=4:1, v v). ¹H NMR (600 MHz, CDCl₃): δ 8.33 (s, 1H), 8.24-8.19 (m, 3H), 7.77 (d, J=8.2 Hz, 2H), 7.44 (s, 1H), 7.41 (t, J=7.8 Hz, 1H), 7.34 (d, J=7.9 Hz, 1H), 2.95-2.90 (m, 1H), 2.58 (s, 3H), 0.93-0.88 (m, 2H), 0.72-0.68 (m, 2H). HRMS: m/z calculated for C₂₁H₁₇F₃N₂O₂S [M+H]⁺: 419.1073; found: 419.0995

### 2. Cell Culture

Human dermal fibroblasts (HDF, PCS-201-010, ATCC, Manassas, VA, USA) were used. The cells were cultured in Dulbecco's Modified Eagle Medium (DMEM) containing 25 mM glucose, supplemented with 10% fetal bovine serum (SH30919.03; Hyclone, Waltham, MA, USA), 100 U/mL penicillin, and 100 μg/mL streptomycin (SV30079.01; Hyclone). Fibroblasts were defined as senescent when their population doubling time exceeded 14 days. Cell viability and cell number were assessed using a Cedex HiRes analyzer (05650216001, Roche, Basel, Switzerland).

### 3. ROS Screening

Senescent fibroblasts were cultured in 96-well plates at a density of 5,000 cells per well. Components from the library were diluted to a final concentration of 4 μM and added to the wells every 4 days. After 12 days of drug treatment, ROS screening was performed. For ROS screening, 100 μL of DHR123 (D23806; Thermo Fisher Scientific) was added per well and cultured for 30 minutes. After the culture, 200 μL of DPBS (20-030-CV; Corning, NY, USA) was added per well, and the wells were washed twice. The washed 96-well plates were then measured using a VICTOR Multilabel Plate Reader (485 nm/535 nm, 0.1 s; 2030-0050; PerkinElmer, Waltham, MA, USA).

### 4. Cell Proliferation Assay

Senescent fibroblasts were cultured in 96-well plates at a density of 1,000 cells per well. Components of the library were diluted to final concentrations of 0-8 μM and added to the wells every 4 days. After 12 days of drug treatment, the cells were washed twice with phosphate-buffered saline (PBS) and lysed in 50 μL of 0.2% SDS. The plates were cultured at 37 °C for 1 hour. GelGreen® nucleic acid gel stain (150 μL) (1:1,000 in DW; 41005; Biotium, Fremont, CA, USA) was added to each well. Fluorescence intensity was measured using a VICTOR Multilabel Plate Reader (2030-0050, PerkinElmer, Waltham, MA, USA) to determine cell number. The mean and standard deviation were calculated from 12 replicates for each experimental group.

### 5. FACS Analysis

FACS analysis was performed using a BD LSR II (BD Biosciences, San Jose, CA, USA) at the Core Facility for Cell-to-In Vivo Imaging. The experimental procedures using FACS are described in detail in a separate paragraph.

### 6. Mitochondrial Membrane Potential (MMP) Measurement

For mitochondrial membrane potential measurement, the cells were cultured with 0.6 μg/mL JC-10 (ab 112134; Abcam, Waltham, MA, USA) at 37 °C for 30 minutes. After staining, measurements were performed using flow cytometry.

### 7. Measurement of ROS, Mitochondrial Mass, and Lipofuscin

For quantification of mitochondrial ROS, cells were cultured in medium containing 3 mM DHR123 (10056-1; Biotium) at 37 °C for 30 minutes. For quantification of mitochondrial mass, cells were cultured in medium containing 50 μM MitoTracker Deep Red (M46753, Thermo Fisher Scientific) at 37 °C for 30 minutes. For lipofuscin measurement in HDF, unstained cells were used. Subsequent measurements were performed using flow cytometry.

### 8. Autophagosome Measurement

Autophagic flux was assessed using CYTO-ID® autophagy detection kit (ENZ-51031-0050, Enzo Lifescience, Farmingdale, NY, USA) according to the manufacturer's protocol, as previously described, and measured by flow cytometry.

### 9. Senescence-Associated β-Galactosidase (SA-β-Gal) Staining

SA-β-gal staining was performed on senescent fibroblasts according to the manufacturer's protocol, followed by observation and analysis.

### Examples

1. Screening for Compounds that Reduce ROS Using High-Throughput Screening

Cellular senescence is characterized by various senescence-associated phenotypes, some of which can be screened in high-throughput assays. Traditionally, high-throughput screening (HTS) has been widely used to identify targets from compound libraries. In the present invention, an HTS method was employed to select for phenotypes with reduced ROS.

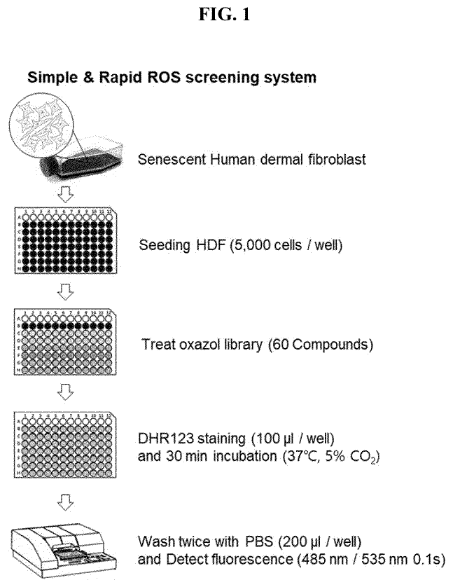

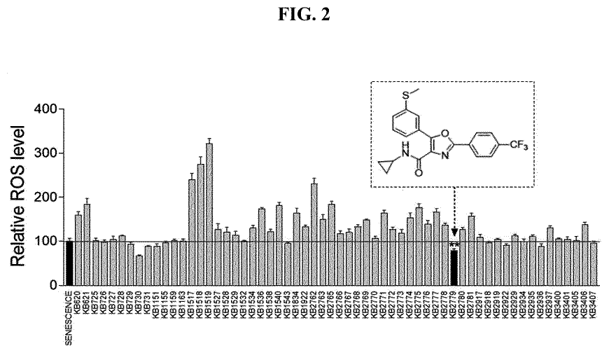

Senescent fibroblasts (with a population doubling time of more than 12 days) were seeded in 96-well plates and treated with oxazole library compounds for 12 days (FIG. 1). After reagent treatment, the medium was removed from the 96-well plates, and 100 μL of cell culture medium containing DHR123 (diluted 1:1,000) was added to each well and cultured for 30 minutes (FIG. 1). After the culture, the 96-well plates were washed twice with PBS, and ROS levels were compared by fluorescence measurement at 485 nm/535 nm (FIG. 1). Among the oxazole library, KB2779 most effectively reduced ROS levels in senescent fibroblasts (FIG. 2). The selected KB2779 suggests potential for senescence improvement.

### 2. Senolytic Effect of KB2779

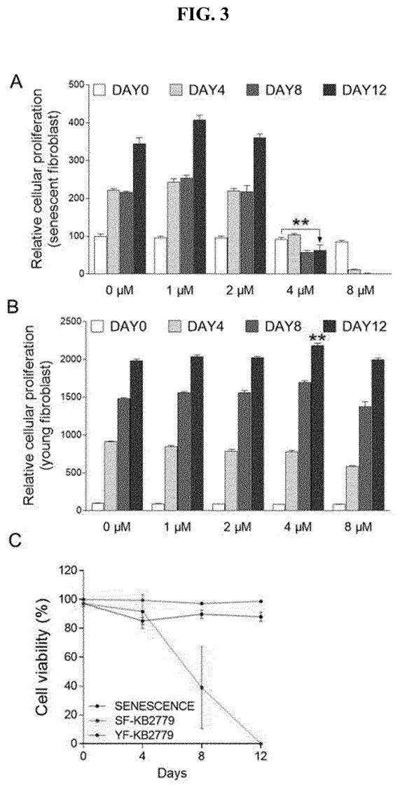

KB2779, identified through ROS screening, can further improve additional senescence-associated phenotypes. Senescent cells are generally known to stop proliferating, and cell proliferation serves as an important indicator of senescence. Therefore, the effect of KB2779 on cell proliferation was observed at various concentrations. The proliferation effect was first observed in senescent fibroblasts (FIG. 2A). At relatively low concentrations (<2 μM), substantial cell proliferation was observed over time (FIG. 3A). However, at relatively high concentrations (>4 μM), a marked decrease in the number of senescent fibroblasts was observed (FIG. 3A). These data indicate that KB2779 inhibits proliferation of senescent fibroblasts at concentrations above 4 μM.

Senolytic therapies are known to selectively induce death of senescent cells, thereby maintaining a youthful cell pool. The present inventors hypothesized that KB2779-mediated suppression of senescent fibroblasts reflects a senolytic effect. Accordingly, the same experiment was conducted on relatively young fibroblasts (population doubling time <2 days) (FIG. 3B).

In young fibroblasts, substantial proliferation was observed over time at all concentrations (FIG. 2B). Even at doses above 4 M, significant proliferation was observed, indicating that KB2779 does not inhibit young fibroblast growth (FIG. 3B). These data suggest that KB2779 exhibits selective suppression in senescent cells at concentrations above 4 μM.

To further confirm the selective inhibitory effect of KB2779, cell viability of fibroblasts was measured over 12 days (FIG. 3C). Young fibroblasts maintained over 95% viability regardless of KB2779 treatment (FIG. 3C). In contrast, senescent fibroblasts showed a significant decrease starting on day 8 after KB2779 administration, with viability dropping to 0% by day 12 (FIG. 3C). These data indicate that KB2779 selectively induces death in senescent fibroblasts.

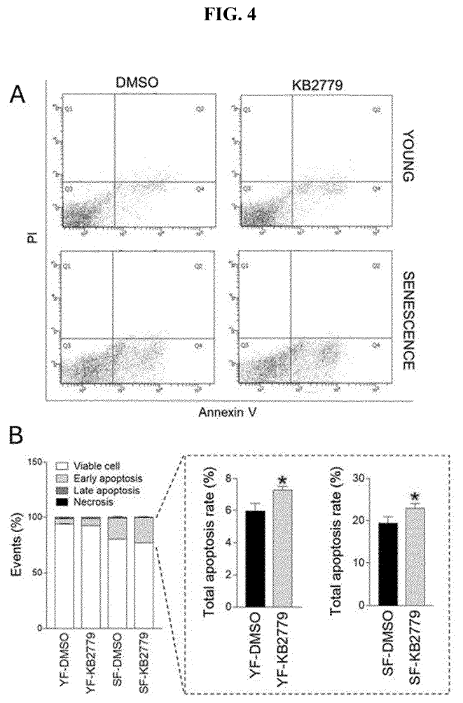

Selective inhibition of senescent fibroblast proliferation provides a basis for the senolytic effect, which occurs through induction of apoptosis in senescent cells. To verify that the selective inhibition of senescent fibroblast proliferation by KB2779 is due to apoptosis, flow cytometry was used to assess cell death rates (FIG. 4A). Apoptosis rates were 6% in young fibroblasts and 7.3% in KB2779-treated young fibroblasts (FIG. 4B). However, in senescent fibroblasts, apoptosis rates were 19.5%, increasing to 23% with KB2779 treatment (FIG. 4B). The significant increase in apoptosis of senescent fibroblasts mediated by KB2779 suggests that its selective inhibitory effect is due to apoptosis.

### 3. Improvement of Senescence-Associated Phenotypes

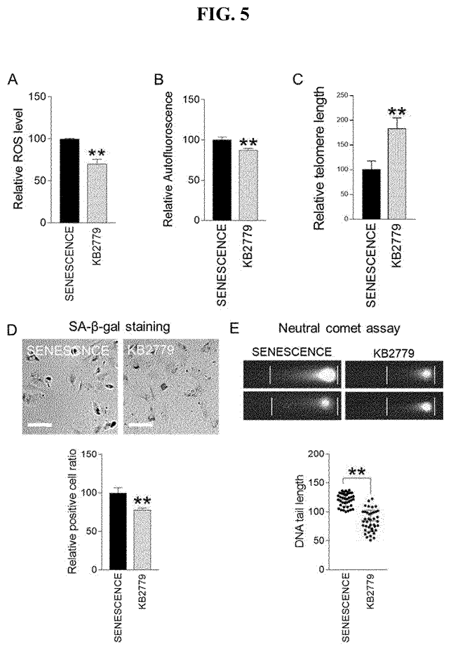

Cellular senescence and accelerated aging are characterized by excessive reactive oxygen species (ROS) generated from dysfunctional mitochondria. In this study, KB2779 was identified through screening as a compound that reduces ROS levels. In FIG. 1, KB2779 decreased ROS levels. To validate this screening result, ROS levels were re-evaluated using flow cytometry. The analysis confirmed that KB2779 actually significantly reduced ROS in senescent fibroblasts (FIG. 5A), thereby verifying the high-throughput screening data shown in FIG. 1.

Lipofuscin is a high-molecular-weight material found in lysosomes, primarily composed of cross-linked protein residues formed through iron-catalyzed oxidative processes. Because it cannot be degraded or eliminated via extracellular excretion, accumulation of lipofuscin within cells is inevitable after mitosis. Lipofuscin accumulates within cells during aging, and a reduction in lipofuscin can serve as an indicator of anti-aging effects. In this study, lipofuscin levels in senescent dermal fibroblasts were quantified using flow cytometry. Treatment with KB2779 significantly reduced the autofluorescence of senescent fibroblasts (FIG. 5B), indicating a decrease in accumulated lipofuscin.

Telomeres, located at the ends of chromosomes, play a crucial role in determining cellular lifespan. Continuous cell division leads to telomere shortening. Treatment with KB2779 restored telomere length in senescent fibroblasts (FIG. 5C), suggesting telomere rejuvenation.

Senescence-associated β-galactosidase (SA-β-gal) accumulates in senescent cells and is a widely used marker for senescence. In the present invention, the proportion of SA-β-gal-positive senescent fibroblasts was markedly reduced following KB2779 treatment (FIG. 5D). This indicates that KB2779 effectively lowers the accumulation of SA-β-gal in the population of senescent fibroblasts.

DNA plays a central role in life processes through replication and transcription. In senescent cells, DNA is often irreversibly damaged, which accelerates aging. Damaged DNA typically appears as small fragments within the nucleus. To assess DNA damage in senescent fibroblasts after KB2779 treatment, a comet assay was performed. The nuclei of untreated senescent fibroblasts appeared substantially enlarged compared to those treated with KB2779 (FIG. 5E). A reduction in nuclear size following KB2779 treatment indicates decreased DNA damage.

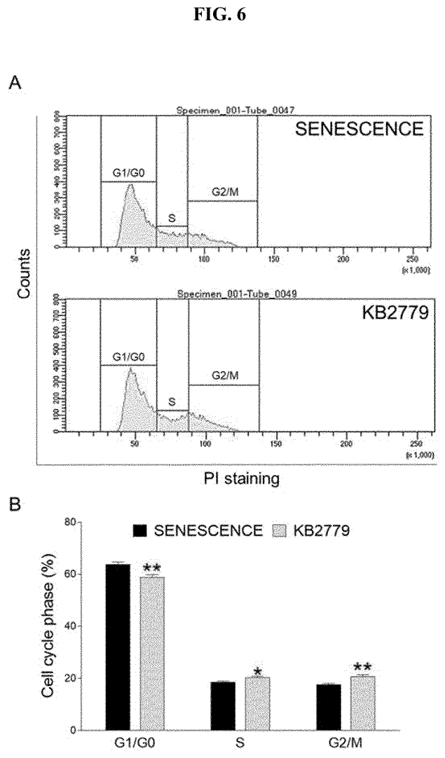

Senescent cells gradually lose their proliferative capacity over time. Cells with reduced division potential typically exhibit cell cycle arrest. To evaluate the effect of KB2779 on the cell cycle of senescent fibroblasts, flow cytometry was performed (FIG. 6A). Untreated senescent fibroblasts showed 63.8% of cells in G1/G0 phase, whereas KB2779-treated senescent fibroblasts displayed a reduced G1/G0 fraction of 58.8%, indicating a notable decrease (FIG. 6B). The proportion of cells in S phase increased from 18.6% in untreated senescent fibroblasts to 20% following KB2779 treatment (FIG. 6B). Similarly, G2/M phase fraction rose from 17.6% to 20% (FIG. 6B). These changes demonstrate that KB2779 treatment reduces G1/G0 arrest and promotes entry into S and G2/M phases, indicating reactivation of the cell cycle in senescent cells.

### 4. Functional Improvement of Autophagosome and Mitochondria

The removal of dysfunctional organelles is one of the critical processes within cells. Traditionally, cells eliminate dysfunctional organelles through the autophagy system. However, as cellular aging progresses, lysosomes and the autophagy system become impaired, preventing normal functioning. The accumulation of dysfunctional organelles accelerates cellular aging.

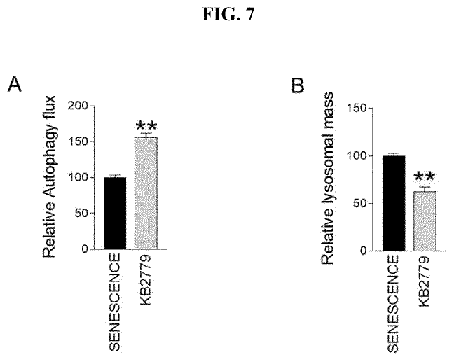

In the present invention, experiments were conducted to determine whether KB2779 improves the autophagy system. Flow cytometry analysis showed that autophagic flux was significantly increased in KB2779-treated senescent fibroblasts (FIG. 7A). Additionally, a significant decrease in lysosomal mass was observed in KB2779-treated senescent fibroblasts (FIG. 7B). These data suggest that KB2779 treatment improves the autophagy system in senescent fibroblasts. This finding indicates the restoration of the lysosome/autophagy system via mitophagy activated by KB2779 in aged fibroblasts.

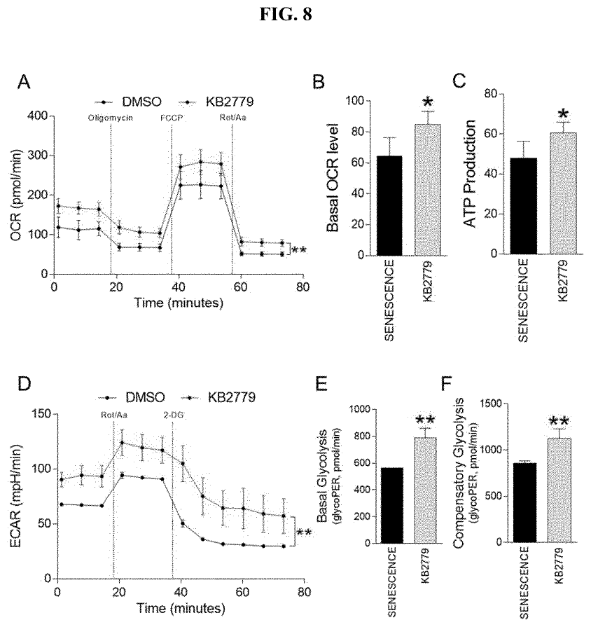

Organelles closely associated with aging are known to be mitochondria. As cells age, damage accumulates in mitochondria, causing dysfunction. It is well established that dysfunctional mitochondria alter cellular metabolism. Considering the enhanced autophagic function, the inventors hypothesized that metabolism in senescent fibroblasts would also change. Accordingly, changes in cellular metabolism induced by KB2779 were observed in fibroblasts. First, KB2779-mediated mitochondrial respiration was measured. A significant increase in the oxygen consumption rate (OCR) was observed in KB2779-treated senescent fibroblasts (FIG. 8A). Substantial increases in basal OCR and ATP production were also observed (FIGS. 8B and 8C). These data indicate KB2779-mediated enhanced mitochondrial metabolism in senescent fibroblasts.

After confirming KB2779-mediated improvement in mitochondrial metabolism, metabolic changes in glycolysis were also observed in KB2779-treated senescent fibroblasts. A significant increase in extracellular acidification rate (ECAR) was observed in KB2779-treated senescent fibroblasts (FIG. 8D). Substantial increases in both basal and compensatory glycolytic activity were also observed (FIGS. 8E and 8F). These data indicate that KB2779 mediates enhanced glycolytic output and potential restoration of glycolytic capacity in senescent fibroblasts. In summary, KB2779 improves the autophagic system and actively modulates cellular metabolic activity.

Although the preferred embodiments of the present invention have been described in detail above, the scope of the present invention is not limited thereto, and various modifications and improvements by those skilled in the art using the basic concepts of the present invention as defined in the following claims also fall within the scope of the present invention.

### <National Research and Development Programs, Supporting the Present Invention>

The present invention was conducted as part of the University-Oriented Research Center Support Program funded by the National Research Foundation of Korea (Grant No. 2020R1A6A1A0304195411).

The present invention was conducted under the Innovative Growth Skin Health-Based Technology Development Program funded by the Ministry of Health and Welfare and supported by the Korea Health Industry Development Institute (Grant No. HP23C0024).

The present invention was conducted as part of the University-Oriented Research Center Support Program funded by the National Research Foundation of Korea under the Ministry of Education (Grant No. 2019R1A6A1A03031807).

What is claim is:

1. An anti-aging composition comprising an oxazole derivative represented by the following [Chemical Formula 1] as an active ingredient;

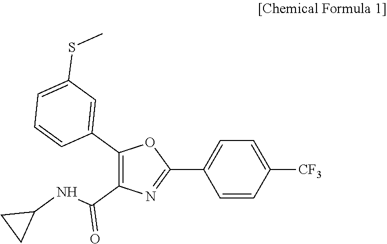 [SMILES: CSc1cccc(-c2oc(-c3ccc(C(F)(F)F)cc3)nc2C(=O)NC2CC2)c1]

2. The composition of claim 1, wherein the aging is induced by mitochondrial dysfunction.

3. The composition of claim 1, wherein the composition reduces reactive oxygen species (ROS) in senescent fibroblasts.

4. The composition of claim 1, wherein the composition enhances intracellular mitochondrial respiration.

5. The composition of claim 1, wherein the composition has a selective cytotoxic effect on senescent cells at a concentration of 4 μM or higher.

6. The composition of claim 1, wherein the composition restores autophagy system and increases glycolytic metabolic activity.

7. A pharmaceutical composition for preventing or treating cellular senescence-related diseases induced by mitochondrial dysfunction, the pharmaceutical composition comprising an oxazole derivative represented by the following [Chemical Formula 1], or a pharmaceutically acceptable salt thereof;

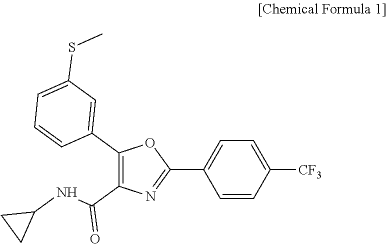 [SMILES: CSc1cccc(-c2oc(-c3ccc(C(F)(F)F)cc3)nc2C(=O)NC2CC2)c1]

8. The composition of claim 7, wherein the senescence-related diseases induced by mitochondrial dysfunction is selected from the group consisting of Friedreich's ataxia (FRDA), renal tubular acidosis, Parkinson's disease, Alzheimer's disease, amyotrophic lateral sclerosis (ALS), Huntington's disease, pervasive developmental disorder, hearing loss, deafness, diabetes, obesity, diabetic ulcer, insulin resistance and hepatic steatosis (steatohepatitis).

9. An anti-aging cosmetic composition comprising an oxazole derivative represented by the following [Chemical Formula 1] as an active ingredient; 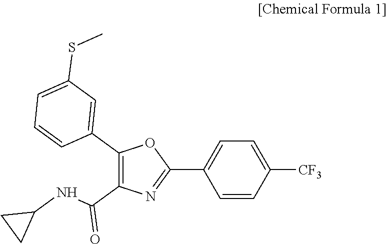 [SMILES: CSc1cccc(-c2oc(-c3ccc(C(F)(F)F)cc3)nc2C(=O)NC2CC2)c1]
